(19) United States
(12) Patent Application Publication
(10) Pub. No.: US 2013/0109078 A1
(43) Pub. Date: May 2, 2013

(54) Methods For Harvesting Biomass

(71) Applicant: Utah State University, North Logan, UT (US)

(72) Inventors: Renil John Anthony, Logan, UT (US); Ronald Sims, Logan, UT (US)

(73) Assignee: Utah State University, North Logan, UT (US)

(21) Appl. No.: 13/663,315

(22) Filed: Oct. 29, 2012

Related U.S. Application Data
(60) Provisional application No. 61/552,604, filed on Oct. 28, 2011.

(51) Int. Cl. C08B 31/00; C12N 1/12

(52) U.S. Cl. 435/257.1; 536/45

(57) ABSTRACT

A method of harvesting biomass in an aqueous solution, where the method includes mixing an organic coagulant or flocculant with a solution comprising biomass and water.

### CROSS-REFERENCE TO RELATED APPLICATIONS

This application claims priority to U.S. Provisional Patent Application No. 61/552,604, filed Oct. 28, 2011, the entirety of which is herein incorporated by reference.

### GOVERNMENT SPONSORED RESEARCH

This invention was made, at least in part, with government support under contract DE-EE0003114 awarded by the United States Department of Energy. The government has certain rights in the invention

### TECHNICAL FIELD

The present disclosure relates to methods of harvesting biomass, more particularly, it relates to methods of harvesting algae with modified starch.

### BACKGROUND

Algae have been identified as a potential biological feed stock in numerous applications. Various methods and/or apparatuses of harvesting algae have been described. For example, in Lijun et al., Preparation and flocculation properties of cationic starch/chitosan crosslinking-copolymer, Journal of Hazardous Materials 172 (1) (December, 2009), the disadvantages of using inorganic coagulants such as alum, ferric chloride and synthetic organic flocculants are described.

Additional and efficient methods for harvesting algae are needed for algae to serve as a large-scale biological feedstock and biomass source.

### SUMMARY

Algae can grow in a variety of environments and conditions. Under suitable growth conditions, microalgae have been shown to double their biomass in 24 hours and in some instances, under exponential growth, the doubling rate is as short as 3.5 hours. The oil content in some species of algae under certain conditions is as high as 80% wt of the biomass. Despite the potential, harvesting or extracting algae from water can be difficult and present a challenge to the ‘algae to biofuels’ program. Methods that have been explored include filtration, centrifugation, evaporation, lypholization, and the use of alum as a coagulant, etc. However, each of these methods may require too much energy, time, or equipment costs to justify the potential biomass harvest.

The present disclosure in aspects and embodiments addresses these various needs and problems by providing a method of harvesting biomass from an aqueous solution, where the method includes mixing an organic coagulant or flocculant with a solution comprising biomass and water.

### BRIEF DESCRIPTION OF THE DRAWINGS

FIG. 1 illustrates the zeta potential of exemplary amino starches.

FIG. 2 illustrates zeta potentials of exemplary amino starches and biomass solutions containing the starches.

FIG. 3 illustrates the % TSS of exemplary amino starches in biomass solution.

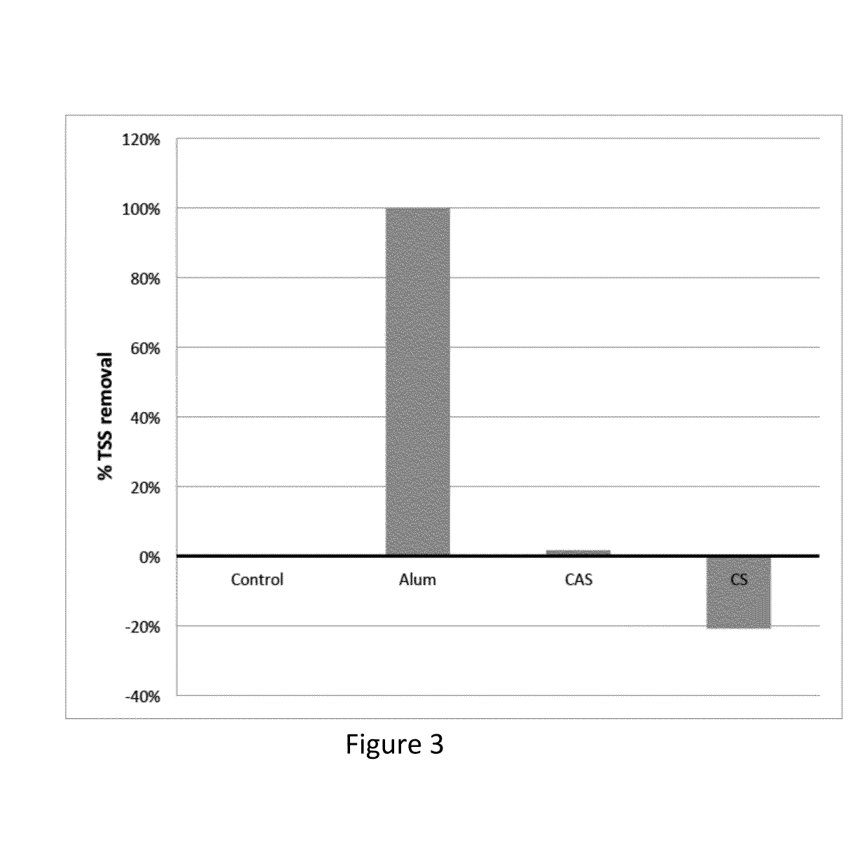

FIG. 4 illustrates the change in zeta potential of exemplary amino starches with pH.

FIG. 5 illustrates the change in zeta potential of exemplary amino starches and aluminum sulphate with pH.

FIG. 6 illustrates the % TSS removal achieved by the addition of exemplary amino starches in a jar test apparatus using microalga Scenedesmus obliquus at pH 7.

FIG. 7 illustrates the % reduction of the zeta potential achieved by the addition of exemplary amino starches in a jar test experiment using microalga Scenedesmus obliquus at pH 7.

FIG. 8 Chromatograms of derivatized (A) Putrescine standard (B) Bacterial putrescine.

### DETAILED DESCRIPTION

The present disclosure covers apparatuses and associated methods for harvesting biomass. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

The present disclosure covers methods, compositions, reagents, and kits for harvesting algae.

In embodiments, organic coagulants and flocculants are employed to effectively harvest algae without negatively affecting the various bio-products that may be later derived from algae. Exemplary bio-products of algae include bio-plastics, biodiesel, bio-solvents, and numerous other products. In embodiments, the organic coagulant and flocculant may comprise a modified starch as described herein.

A. Starch

Starch is an abundant natural polymer available from sources such as potato, corn, rice, tapioca, etc. Irrespective of the source, starch is primarily comprised of amylose (20-30% wt) and amylopectin (70-80% wt), which are illustrated below:

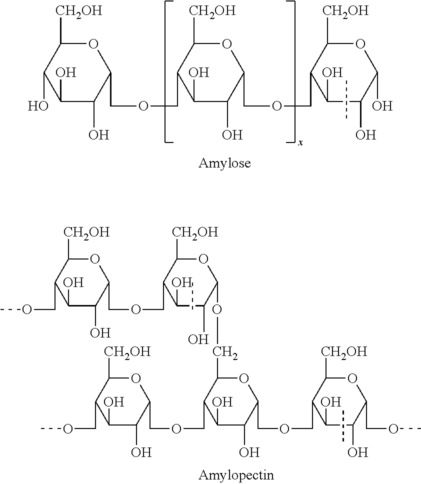

In some embodiments, the starch source may be what would otherwise be considered a waste product, such as waste starch derived from potato, or other vegetable, processing.

The starch may be modified to have cationic groups, such as amine, ammonium, phosphonium, or imines. By modifying the starch with cationic groups, the starch may then serve as an organic coagulant and flocculant for algae harvesting.

B. Amines and Amine Extraction

Any suitable amine or mixture of amines may be used as a cationic group to modify the starch. For example, at least one of a primary, secondary, tertiary amines, or quaternary ammonium may be used.

In some embodiments, one source of amines includes decomposing organic matter, such as fish waste, which generate amines by the decarboxylation of amino acids. Naturally, decarboxylation occurs by enzyme activity of bacteria (Enterobacteriaceae, Enterococci, etc.) on amino acids. For example, the following amines and parent amino acids are listed below:

| Amino acid | Amine | Structure |
|---|---|---|
| L-ornithine | Putrescine | 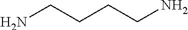 |
| L-lysine | Cadaverine | 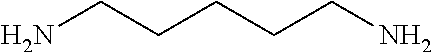 |
| L-histidine | Histamine | 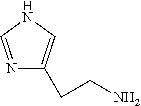 |
| Tyrosine | Tyramine | 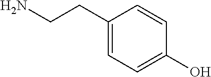 |
| L-ornithine | Spermidine | 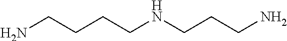 |

Amines may be removed from amine sources by mixing the source material with a solvent, such as methanol. Suitable amines described herein may include biogenic amines, which may be derived from any natural source that comprises amino acids or proteins. In some embodiments, biogenic amines from fish, fish waste, meat, and meat waste may be used. For example, the source may be blended to a coarse paste to facilitate quick decomposition. Amine extraction from decomposing material, such as fish, may be carried out with any suitable solvent. Exemplary solvents include methanol, trichloroacetic acid, perchloric acid, and water.

Exemplary extraction procedures may include mixing an optionally blended or otherwise mechanically broken down protein or amino acid source with a solvent; optionally heating the slurry to a suitable temperature, such as from about 30° C. to about 150° C., from about 50° C. to about 70° C., or about 60° C., for a suitable period of time, such as from about 1 minute to about 2 hours, from about 10-90 minutes, or about 15 minutes. The time and temperatures may vary depending on the source material solvent concentrations. After mixing and optional heating, the liquid may be separated from the solid by suitable solid-liquid separations methods, such as filtration, centrifugation, etc. The supernatant is removed and may be stored at 0° C. until further use.

Amines may also be generated from hydrolyzed fish or other waste containing proteins by bacteria in a controlled environment. Niven's media may be modified by the exclusion of agar and histidine. The amino acid histidine may be replaced with hydrolyzed fish waste and incubated at 25° C. to about 40° C. for 4-10 days. The resulting mixture may then be centrifuged and the supernatant stored at 0° C. until further use.

C. Starch Modification

The starch may be modified by any suitable method. In some embodiments, the starch is modified by halogenation followed by alkylation of amine with a halogen-starch, as set forth in the following reaction scheme:

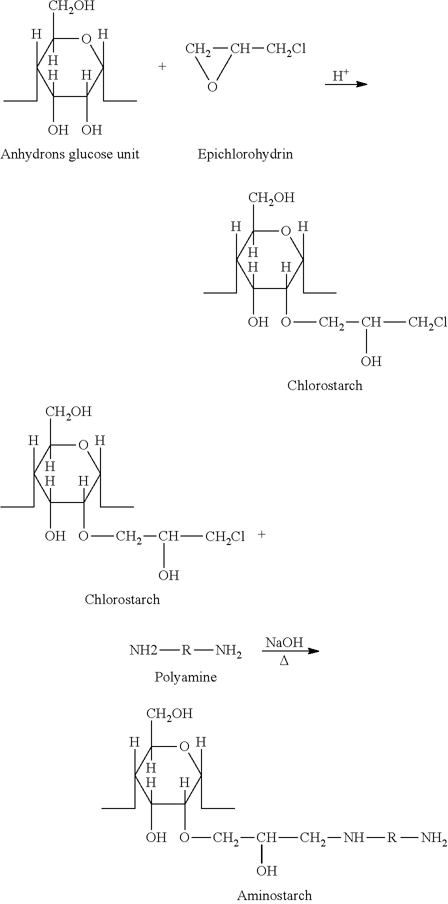

The halogenation and alkylation reactions may employ other reactants. For example, in the halogenation reaction, a halogenating reactant such as phosphorus pentachloride may be used as reactant and an acid, such as hydrochloric acid as catalyst. For the alkylation reaction, any form of amine such as primary, secondary or tertiary amine may be used as reactants in a basic solution with a pH of about 8 to 13.

To begin with, corn starch may be crosslinked by mixing it with epichlorohydrin in a basic solution, such as a 1 N NaOH solution with a pH to from about 8 to about 13 for about 15 to about 24 hours at room temperature. After crosslinking, the starch may be separated from solution by centrifugation, or other suitable solid-liquid separation method, at, for example, 5000 rpm for a period of time, such as 2 minutes at room temperature. The separated starch may then be mixed with epichlorohydrin and perchloric acid for 30 minutes at 60° C. to be prepared as halogenated/chlorinated starch. The halogenated/chlorinated starch may then be reacted with the desired amine for 8 to 10 hours at, for example, 60° C. in a basic solution with a pH of from about 8 to about 13 resulting in Cationic Amino Starch (CAS).

This modified starch may be separated from the solution by precipitation with, for example, ethanol. The solution, may be centrifuged, or otherwise subjected to a solid-liquid separation technique, to collect the precipitate and the supernatant may then be discarded. The precipitate may be washed with a suitable washing agent, such as ethanol in a soxhlet apparatus with a reflux time which may include up to 20 hours, such as about 5 to 15 hours, or about 12 hours to clean the starch of any unreacted reagents and catalyst. The modified starch may then be dried of the washing agent, optionally pulverized, and stored at room temperature until further use.

Amines are basic and this basicity makes them readily available for nucleophilic substitution with alkyl halides under mild conditions. After initial alkylation, depending upon the amount of reactants present in excess, subsequent alkylation may result, leading to tertiary amines and then quarternary ammonium.

After modified starch preparation, the zeta potential may be measured to examine the potency of the modified starch as a potential coagulant and flocculant. Zeta potential is the measure of charge present on a colloidal particle surface. For the modified starch to show cationization, the zeta pontential should be greater than 0. Minimum zeta potential above about +8 mV is necessary for the feasibility of starch as a coagulant/flocculant for algae separation and harvesting. Suitable zeta potentials for the modified starch as a coagulant/flocculant may include, for example, from about +3.8 to about −7.0 mV in a pH of about 5.0 to about 10.0; or from about +5.5 to about +0.5 mV in a pH of about 5.0 to about 10.0.

Degree of substitution (DS) relates to the number of hydroxyl groups (maximum 3) on one anhydrous glucose unit of starch that are substituted by amines (N-group). In embodiments, the higher the degree of substitution, the greater would be the neutralizing capability of a modified starch resulting in efficient separation with minimal dosage. Suitable DS values may include, for example, from about 0.0083 to 0.57

D. Precipitate Formation

The CAS, or modified starch, may be mixed with an aqueous solution containing algae to be harvested. Suitable ratios include, for example, from about 0.5:5 to 5:0.5 starch:algae, such as from about 0.75:1.25 to 2:0.75, from about 1:0.75 to 0.75:2, or about 1.5:1. Upon addition of the modified starch, the solution may be optionally flash mixed to facilitate uniform mixing of the modified starch in the suspension for charge neutralization and to avoid lump formation. Flash mixing may be followed by slow mixing to facilitate bridging (particle interaction between algae and starch) of the neutralized algae particles and also to help in residual charge neutralization not achieved by flash mixing. The mixing may be then stopped and the flocs are allowed to sediment for a period of time. Precipitate formation may be performed in a suitable reactor equipped with optional stirrers and/or convection properties.

The following examples are illustrative only and are not intended to limit the disclosure in any way.

### EXAMPLES

### Example 1

### Preparation Cationic Amino Starch (CAS) and Chlorostarch (CS)

In the preparation of CAS, the first step was to prepare crosslinked starch by reacting 10 g of starch with 800 uL of epichlorohydrin in 1 N NaOH solution in 200 ml DI water for 20 hours at room temperature. The crosslinked starch was separated from solution by centrifugation at 5000 rpm for 2 mins at 25° C. The next step was the preparation of chlorostarch, by reacting the crosslinked starch with 10 ml of epichlorohydrin and 180 ul of perchloric acid for 30 minutes at 60° C. After the prescribed time period, 0.5 g of putrescine was added to the chlorostarch solution and reacted for 8 hours in 0.16 N NaOH solution. Further, the starch was precipitated out of solution using ethanol as needed. The solution was then centrifuged at 5000 rpm for 5 minutes at 25° C. to separate the precipitate formed. The precipitate was then washed with ethanol in a soxhlet apparatus with a reflux time of 8 hours to clean the CAS of any unreacted reactants or reagents. After washing, the CAS was dried of ethanol, pulverized, and stored until further use.

To study the effect of putrescine on the preparation of amino starch, a control was prepared as follows: In the preparation of CS, the first step was to prepare crosslinked starch by reacting 10 g of starch with 800 uL of epichlorohydrin in 1 N NaOH solution in 200 ml DI water for 20 hours at room temperature. The crosslinked starch was separated from solution by centrifugation at 5000 rpm for 2 mins at 25° C. The next step was the preparation of chlorostarch, by reacting the crosslinked starch with 10 ml of epichlorohydrin and 180 ul of perchloric acid for 30 minutes at 60° C. in 0.16 N NaOH solution. The chlorostarch solution continued reaction for 8 hours in 0.16 N NaOH solution. Further, the starch was precipitated out of solution using ethanol as needed. The solution was then centrifuged at 5000 rpm for 5 minutes at 25° C. to separate the precipitate formed. The precipitate was then washed with ethanol in a soxhlet apparatus with a reflux time of 8 hours to clean the CS of any unreacted reactants or reagents. After washing, the CS was dried of ethanol, pulverized, and stored until further use.

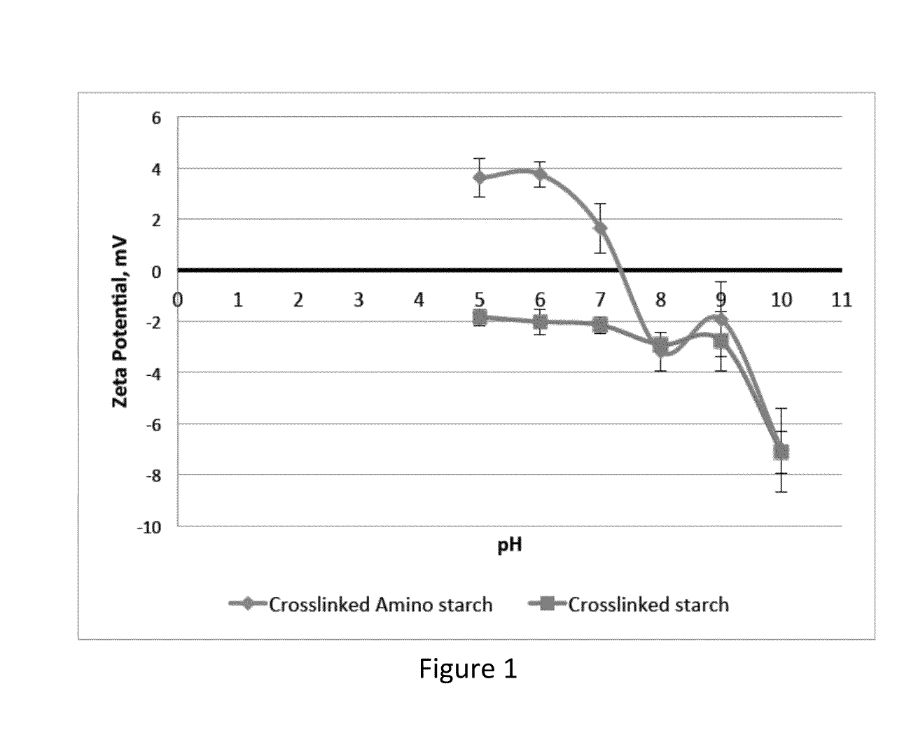

The zeta potential for CAS and CS was measured with varying pH (5 to 10). For CAS, due to the N-group attached to the starch molecule, as the pH is varied from 10 to 5 we can see, with the help of the change in zeta potential, the protonation of the N-group on the starch molecule as the pH is reduced from basic to acidic. For pH 7 and lower, protonation of the N-group takes place and thus results in positive zeta potential. However, for CS lacking N-group shows no change in zeta potential with varying pH and behaves like a typical negatively charged biological particle. This experiment illustrates the difference in zeta potential behavior with varying pH for CAS and also the difference between CAS and CS with respect to zeta potential. As is illustrated in FIG. 1, as the pH increased, the zeta potential of CAS varied and shifted from positive to negative. Thus pH may be used to control the charge of CAS.

The total nitrogen content of CAS and CS was determined using a Hatch Total N kit in order to determine the degree of substitution. The degree of substitution was calculated using the following formula:

$$D S = \frac{162 \% N}{[ 2800 - 88.5 \% N ]}$$

162=M.W. of starch; 88.5=M.W. of putrescine; % N=% of total N in starch. The degree of substitution is a measure of substitution of the hydroxyl group in one anhydrous glucose unit of starch. One anhydrous glucose unit of starch contains 3 hydroxyl groups. Hence, the maximum degree of substitution that a modified starch can attain is 3. CAS had a DS of 0.0025 and CS had a DS of 0. This test confirms the attachment of N-group to the starch molecule in CAS and shows no nitrogen in CS as a result of no putrescine addition in the preparation stage.

### Example 2

### Jar Test

A contol batch was prepared and refers to 1 liter of algal suspension (concentration ˜20 mg/L) without any coagulant and/or flocculant addition at pH 7 using microalga Scenedesmus obliquus. This jar was used as a control for the jar test experiment. The control is referenced as jar no. 1.

An inorganic batch was prepared with the same parameters as the control but with 10 mg of alum added. The alum solution was received from Tatcher chemicals with a concentration of 376 g/L which was then diluted to a workable concentration. This batch is referenced as jar no. 2.

A CAS batch was prepared using the CAS prepared in Example 1. About 10 mg of CAS were added to a 1 liter algae solution with concentrations of algae as in the inorganic and control batches. This batch is referenced as jar no. 3 and had a ratio of starch to algae of about 1:1.

A CS batch was similarly prepared as was the CAS batch, except that 10 mg of CS, as prepared in Example 1 were used. This batch is referenced as jar no. 4.

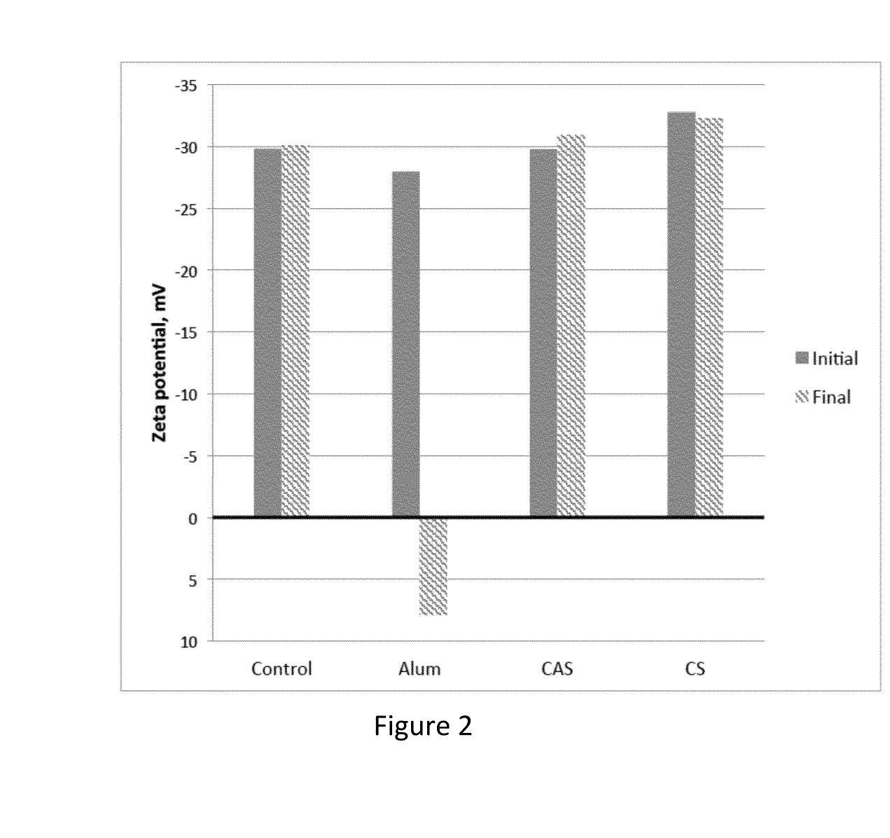

For the jar test two parameters were measured: (1) zeta potential reduction of the algae culture and (2) % Total suspended solids removal. For the control batch (jar 1), no zeta potential reduction or % TSS removal was observed. For the alum batch (jar 2), it was observed that due to the excess of alum, the overall negative charge on the algae suspension was neutralize over and above driving the zeta potential of the suspension to a positive value. About 100% TSS removal was observed with alum addition due to successful charge neutralization. For jar 3 with CAS, no significant reduction in zeta potential was observed. This could be due to the inherently low positive zeta potential on CAS requiring a higher dosage to achieve charge neutralization of algae. No significant TSS removal was observed as well. For jar 4 with CS, no reduction in zeta potential was observed, this was expected as CS has a negative zeta potential inherently incapable of reducing the zeta potential of algae. TSS measurement of jar 4 showed higher TSS than initial. This is due to the starch particles in suspension. The results are illustrated in FIGS. 2 & 3

### Example 3

### Preparation of PCAS

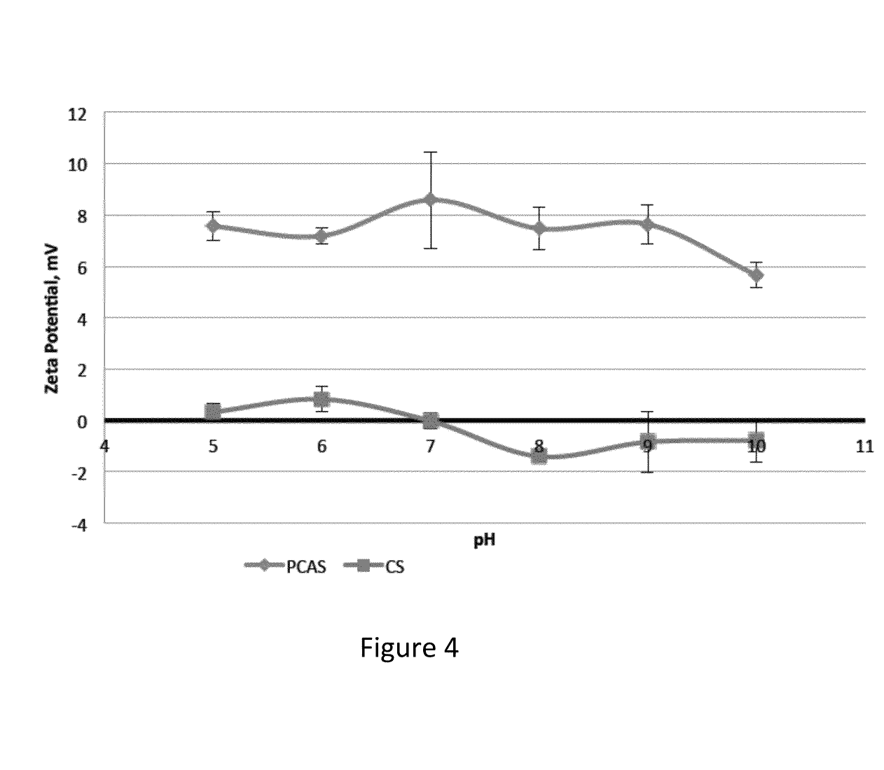

The method described in Example 1 was again used, this time using 5g of corn starch and 800 ul of epichlorohydrin to prepare crosslinked starch. To prepare chlorostarch 5 ml of epichlorohydrin and 0.5 ml of HClO₄ were used. (60%) was reacted as described in Example 1. Further, 0.5 g of putrescine was added to the chlorostarch to yield a further substituted modified starch. The DS was again measured for the intermediate product CS, which was 0, and for PCAS, which was 0.0083. The zeta potential was again measure at varying pHs. The results are illustrated in FIG. 4, which show that when the PCAS is more substituted, the pH does not affect the charge to the same degree.

### Example 4

### Preparation of HCAS

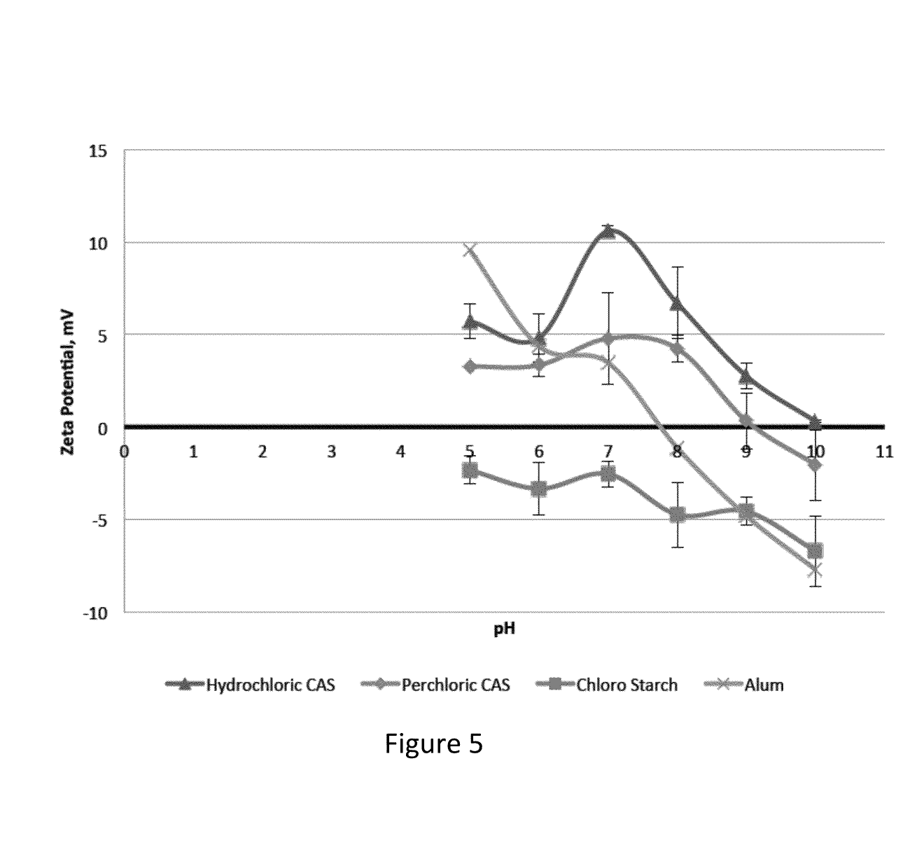

The method described in Example 6 was again used, this time using 0.5 ml HCl, instead of HClO₄, to yield a modified starch referred to as HCAS. Perchloric acid was substituted with HCl due to the hazards associated with the use of the former. The DS for HCAS was 0.0151. The zeta potential was again measure at varying pHs for PCAS, HCAS, CS and alum. It was observed that the zeta potential of PCAS and HCAS remained fairly positive across the entire range of pHs tested. One explanation includes formation of quarternary ammonium from primary amine by multiple alkylation due to the presence of excess catalyst (HCl or HClO4) which helps in the formation of chlorostarch which in turn undergoes alkylation with amines. Quarternary ammonium is protonated at all pHs and hence we see positive zeta potential for PCAS and HCAS for all the pH values tested. The results are illustrated in FIG. 5 along with that of PCAS, CS, and alum.

### Example 5

### Jar Test 2

Jar tests, similar to those described above, were performed with the synthesized starches PCAS, HCAS and CS using microalga Scenedesmus obliquus. Jar tests were carried out one at a time with different starches. The jar tests were based on standard methods as described in Precipitate Formation. TSS measurements were conducted using 2540 D standard method. Out of the 6 jars (algae concentration ˜60 mg/L), jar 1 was the control without addition of any coagulant or flocculant. Jars 2 to 6 had concentrations of 25, 50, 100, 200 and 300 mg/L of PCAS, respectively. The pH was maintained at 7. Similar procedure was followed for HCAS and CS. The zeta potential reduction after adding the starches were also measured for all the three jar tests.

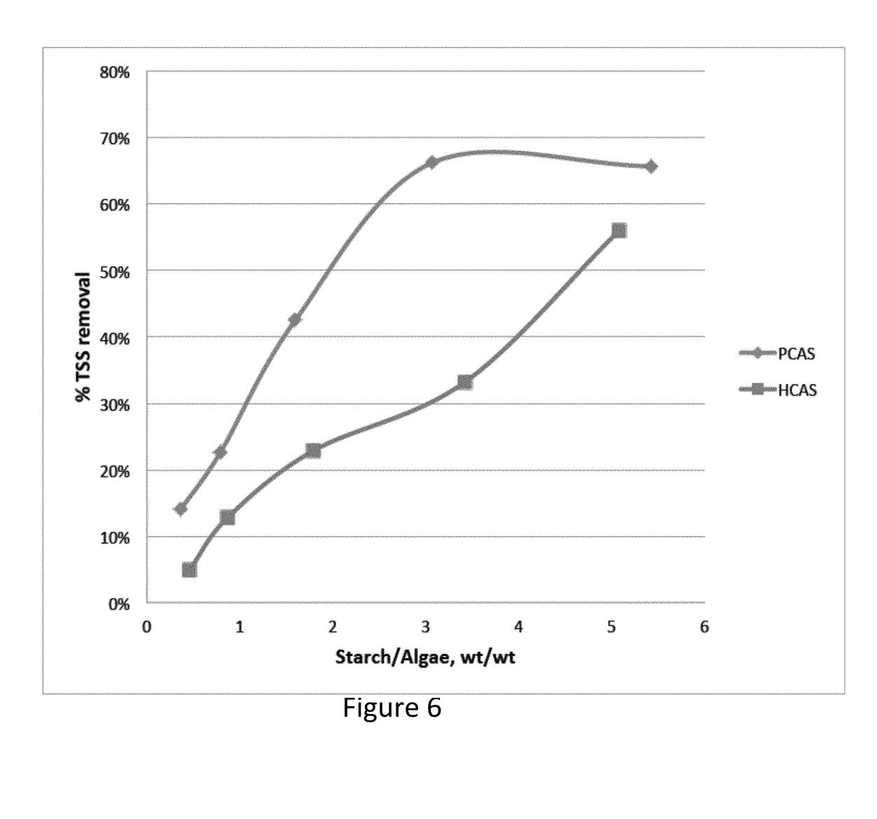

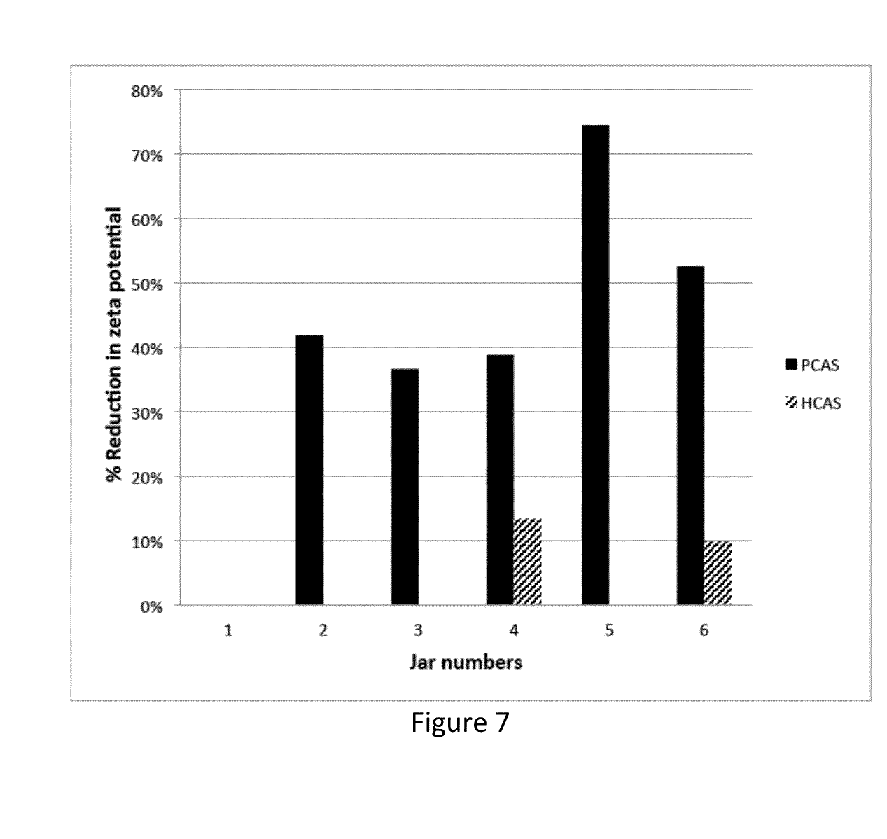

Jar test results show over 60% TSS removal when PCAS is used at a weight ratio of 3:1 (Starch: Algae) and above achieving a reduction in zeta potential upto 70%. The results for the jar test using HCAS do not match in terms of % TSS removal and reduction in zeta potential. This could be due to the difference in sampling equipment for HCAS (specifically different make cuvettes for zeta potential measurement). However, HCAS shows % TSS removal of close to 60% with weight ratio of 5:1 (Starch: Algae). The reduction in zeta potential for this % TSS removal is merely 10% which does not match. This value should have read higher to achieve a % TSS removal of 60%. In comparing the HCAS and PCAS batches, FIGS. 6 and 7 illustrate some of the differences.

### Example 6

### Bacterial Production of Amines

Isolation of bacteria: For the isolation of amine producing bacteria, modified Niven's media was prepared containing 0.5% Tryptone, 0.5% yeast extract, 2.7% L-ornithine, 0.5% NaCl, 0.1% CaCO3, 2.0% agar and 0.006% phenolphthalein (pH 7.0) by weight. The media was autoclaved for 10 mins after which, it was poured onto the plates and allowed to solidify. After solidification, 250 ul of tuna extract was plated and duplicate plates were incubated at 25° C., 30° C. and 37° C. for 72 hours. After incubation, the bacterial colonies were examined for the surrounding pink halo. The color change for original (yellow) to pink was the result increase in pH by at least 1.5 pH units due to the accumulation of the alkaline putrescine. The bacterial colonies grown at 30 oC were isolated and grown in suspension in modified Niven's media without the agar.

Putrescine production by bacteria: The bacteria was grown in modified Niven's media at 30° C. for 96 hours. Duplicate flasks with bacteria were grown with the phenolphthalein indicator, one flask was used as the control (with the indicator and without the bacteria), the fourth flask which served as the freezer stock for later experiments contained the media and the bacteria without the phenolphthalein indicator. After 96 hours, the flasks with the bacteria and the phenolphthalein indicator turned pink from yellow indicating putrescine formation and showed a pH of 9.5, indicating an increase in 2.5 units of pH. The control did not change color and maintained the original yellow color with the starting pH at 7.00. The contents in the pink flasks were centrifuged and analyzed on GC for identification and quantification of compounds.

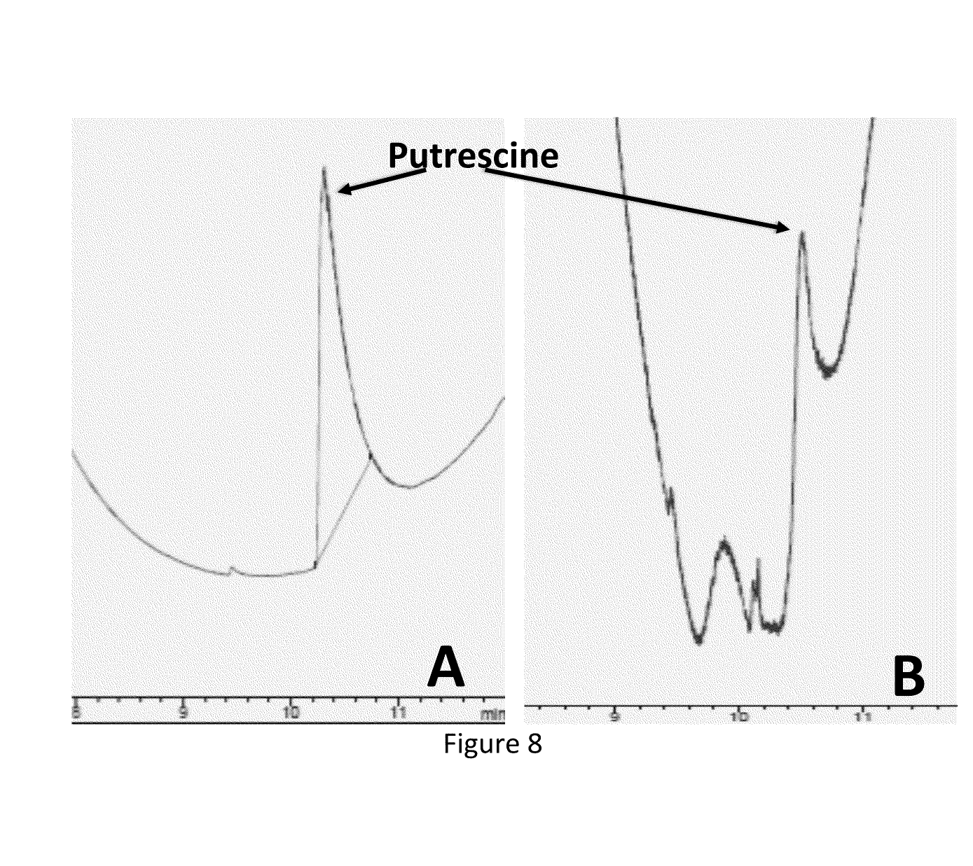

Analysis of putrescine: The 50 ml of the supernatant was derivatized by adding 2 ml of propyl chloroformate and vortexing for one minute. The mixture was centrifuged and the supernatant was discarded and the pellet dissolved in 2 ml chloroform by vortexing for one minute. The mixture was again centrifuged and the supernatant was placed in GC vials for analysis. The bacterial putrescine was compared against a derivatized putrescine standard to detect 300 mg/L of bacterial putrescine in the sample (FIG. 8).

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A method of harvesting biomass in an aqueous solution, the method comprising:
mixing an organic coagulant or flocculant with a solution comprising biomass and water.

2. The method of claim 1, wherein the biomass comprises algae.

3. The method of claim 1, wherein the organic coagulant or flocculant comprises an amine-modified starch.

4. The method of claim 3, wherein the amine-modified starch comprises an amine group derived from a primary, secondary, or tertiary amine or a quaternary ammonium.

5. The method of claim 3, wherein the amine-modified starch comprises a cationic group derived from a quaternary ammonium.

6. The method of claim 1, wherein the organic coagulant or flocculant has a zeta potential of from about +3.8 to about −7.0 mV in a pH of about 5.0 to about 10.0.

7. The method of claim 3, wherein the amine-modified starch comprises an amine group derived from organic waste.

8. The method of claim 7, wherein the organic waste is fish waste.

9. The method of claim 1, wherein the organic coagulant or flocculant is produced by a method comprising:
halogenating a starch to form a halogen-starch, and
alkylating an amine with the halogen-starch to yield a modified starch.

10. The method of claim 9, wherein halogenation occurs in the presence of perchloric acid or hydrochloric acid.

11. An organic coagulant, comprising an amine-modified starch.

12. The organic coagulant of claim 11, wherein the amine-modified starch comprises an amine group derived from a primary, secondary, or tertiary amine, or quaternary ammonium.

13. The organic coagulant of claim 11, wherein the amine-modified starch comprises a cationic group derived from a quaternary ammonium.

14. The organic coagulant of claim 11, wherein it has a zeta potential of from about +5.5 to about +0.5 mV in a pH of about 5.0 to about 10.0.

15. The organic coagulant of claim 11, wherein the amine-modified starch comprises an amine group derived from organic waste.

16. The organic coagulant of claim 15, wherein the organic waste is fish waste.
